(19) United States
(12) Patent Application Publication
(10) Pub. No.: US 2025/0375353 A1
(43) Pub. Date: Dec. 11, 2025

(54) CONTAINER FOR FLUID FOR DELIVERY TO A PATIENT

(71) Applicant: Fresenius Medical Care Holdings, Inc., Waltham, MA (US)

(72) Inventors: Diego Suarez del Real Pena, Mission, TX (US); Jose Omar Aguilar Flores, Mission, TX (US); Irving Uziel Hernandez Gomez, Hidalgo, TX (US)

(21) Appl. No.: 18/740,190

(22) Filed: Jun. 11, 2024

(51) Int. Cl. A61J 1/14; A61J 1/05

(52) U.S. Cl. CPC A61J 1/1468; A61J 1/05; A61J 2200/76

(57) ABSTRACT

The present teachings generally include devices, systems, kits, and methods related to containers for a liquid solution intended for transport into a patient's body—e.g., via an intravenous (IV) treatment, a dialysis treatment, and the like. A container of the present teachings may be structurally configured to be relatively easy to lift and handle, transport, and/or use. Moreover, a container of the present teachings may be sized and shaped to promote grouping of multiple containers together into a predetermined group shape, which can be beneficial for packaging, handling, and/or transport. To this end, a container of the present teachings may include substantially planar sidewalls and a base featuring a connector that projects downwardly along a z-axis no further than terminal ends of sidewalls disposed adjacent to the base, where a cross-section of the container approximates a polygon to promote the grouping of multiple containers together for packaging and transport.

### FIELD

The present disclosure generally relates to devices, systems, kits, and methods featuring containers for fluids to be delivered to a patient, for example, for use in a dialysis treatment, an intravenous (IV) delivery system, and/or the like.

### BACKGROUND

Many medical treatments include formulated fluids entering the human body, e.g., through a vein or through a catheter to the peritoneal cavity. By way of example, in peritoneal dialysis, a dialysis solution with sodium, calcium, magnesium, chloride, and lactate flows from a plastic bag through a catheter into a patient's abdomen. By way of further example, in hemodialysis, a sodium chloride solution (a saline solution) is used to prime a tubing set and dialyzer, ensuring that the tubing is free of air bubbles and ready for administering medications or fluids directly into the patient's bloodstream via an intravenous (IV) line, e.g., by administering fluid from a dialysate bag or the like. In this manner, the aforementioned example treatments may include the use of dialysate bags and/or IV bags, which can include flexible containers to deliver fluids, medications, and/or nutrients directly into a patient's body (e.g., into the patient's bloodstream through a vein) or to a dialyzer or similar.

Such bags are typically made of sterile plastic and come in various sizes, usually ranging from 50 milliliters to several liters. For example, dialysate bags are typically packaged in a plastic overwrap and sterilized via an autoclave or the like. The overwrap is used to contain fluid in the event of bag leakage, e.g., protecting other products included in the same packaging box. Automated peritoneal dialysis (APD) bags are typically packaged in sets of two, four, or six per carton box and hemodialysis (HD) bags are typically packaged in sets of 12 per carton box. The lifting and hanging of these bags (e.g., to an IV pole) can be difficult due to the odd shape of the bags, which often lack a feature for grasping. Also, the shipment of filled plastic bags can be problematic, as they may need to be handled with care to prevent leakages. In addition, the use of an overwrap can add extra material costs to the bags. Finally, the packaging design of existing bags is less than optimal because there can be air gaps inside the carton cases that weaken the integrity of the cases when they are shipped in pallets, while also generating costs associated with the quantity of bags that can be packaged in a pallet due to wasted space inside the carton cases.

There remains a need for improved containers for fluids to be delivered to patients, e.g., in an IV treatment, a dialysis treatment, and the like.

### SUMMARY

The present teachings generally include devices, systems, kits, and methods related to containers for a liquid solution intended for transport into a patient's body—e.g., via an intravenous (IV) treatment, a dialysis treatment, and the like. A container of the present teachings may be structurally configured to be relatively easy to lift and handle, transport, and/or use. Moreover, a container of the present teachings may be sized and shaped to promote grouping of multiple containers together into a predetermined group shape, which can be beneficial for packaging, handling, and/or transport. To this end, a container of the present teachings may include substantially planar sidewalls and a base featuring a connector that projects downwardly along a z-axis no further than terminal ends of sidewalls disposed adjacent to the base, where a cross-section of the container approximates a polygon to promote the grouping of multiple containers together for packaging and transport.

These and other features, aspects, and advantages of the present teachings will become better understood with reference to the following description, examples, and appended claims.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 shows a top, rear perspective view of a container for holding a liquid solution intended for transport into a body of a patient, in accordance with a representative embodiment.

FIG. 2 shows a front view of a container, in accordance with a representative embodiment.

FIG. 3 shows a bottom perspective view of a container, in accordance with a representative embodiment.

FIG. 4 shows a side, bottom perspective view of a container, in accordance with a representative embodiment.

FIGS. 5A-5D show cross-sectional views through a container, in accordance with a representative embodiment.

FIG. 6 shows a container in use, in accordance with a representative embodiment.

FIGS. 7A and 7B show fluid delivery systems, in accordance with representative embodiments.

FIGS. 8-11 show a plurality of containers, in accordance with representative embodiments.

FIG. 12 is a flow chart of a method of manufacturing a container, in accordance with a representative embodiment.

### DETAILED DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately” or the like, when accompanying a numerical value, are to be construed as indicating a deviation as would be appreciated by one of ordinary skill in the art to operate satisfactorily for an intended purpose. Similarly, words of approximation such as “about,” “approximately,” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” and the like, are words of convenience and are not to be construed as limiting terms unless specifically stated to the contrary.

The present teachings generally include devices, systems, kits, and methods related to containers for holding a liquid solution intended for transport into a patient's body—e.g., via an intravenous (IV) treatment, a dialysis treatment, and the like. More specifically, a container disclosed herein may be specifically designed to be relatively easy to lift and handle, transport, and/or use, in comparison with standard fluid delivery bags (e.g., IV bags). To this end, a container disclosed herein may be sized and shaped to promote grouping of a plurality of containers together into a predetermined group shape—e.g., a shape approximating a rectangular box or similar—which can be beneficial for packaging, handling, and/or transport. Therefore, a container of the present teachings may include substantially planar sidewalls and a base featuring a connector that projects downwardly along a z-axis no further than terminal ends of sidewalls disposed adjacent to the base, where a cross-section of the container approximates a polygon to promote the grouping of multiple containers together for packaging and transport. In certain aspects, containers according to the present teachings can be used in medical treatments instead of standard IV bags, dialysis bags, and similar, where existing versions of these items may be frailer and more cumbersome than the containers described herein.

A container disclosed herein may be used in a medical treatment such as one involving administering fluids, medications, and/or nutrients directly into a patient's vein using a needle or a catheter—i.e., an IV treatment. That is, containers according to the present teachings may be suitable for use in IV treatments in various medical settings—e.g., hospitals, outpatient clinics, and the like—and for a wide range of purposes such as those including, without limitation, one or more of the following: hydration, e.g., administering fluids to treat dehydration caused by illnesses, strenuous exercise, and/or inadequate fluid intake; medication delivery, e.g., delivering antibiotics, chemotherapy drugs, pain relief medications, and/or other drugs that require rapid and/or controlled administration; electrolyte balance, e.g., to address imbalances of electrolytes such as sodium, potassium, and magnesium; nutritional support, e.g., providing nutrients for patients who cannot eat or absorb nutrients orally, known as total parenteral nutrition (TPN); blood transfusions; immunotherapy; therapeutic phlebotomy, e.g., removing blood from the body to treat conditions like hemochromatosis, where excess iron is present; and the like.

A container disclosed herein may be used in a dialysis therapy, treatment, or procedure, e.g., one or more of hemodialysis and peritoneal dialysis. That is, a container of the present teachings may contain dialysate or the like that is used in such procedures/treatments. Existing bags used for, e.g., automated peritoneal dialysis (APD) and hemodialysis (HD), are often difficult to ship, store, lift, and hang, which can be due to the relatively odd shape of the bags, especially when filled, and because these bags often lack handles, grips, or similar. In addition, many such bags include an overwrap, which adds extra material costs and waste to the product. Finally, the packaging design of existing bags may be suboptimal—when packaged in a case, there can be air gaps that weaken the shipment. Moreover when existing bags are shipped in pallets, there are often wasted costs associated with the quantity of bags that can be packaged in a pallet due to unused space inside packaging due to the bags' design and/or from support material needed to prevent the bags from breaking or leaking.

The present teachings may include embodiments that solve many of the aforementioned issues with existing IV bags, dialysate bags, and similar. In some aspects, this includes a container (e.g., for IV and/or dialysis therapy) that: is relatively easy to lift (e.g., compared to existing IV bags, dialysate bags, and similar), optimizes a packaging space for such containers (e.g., by being configurable into a shape that conforms to a box/package when grouped with other containers), and is relatively easy to handle, transport, and use (e.g., compared to existing IV bags, dialysate bags, and similar).

In general, in some aspects, the present teachings include a container formed in a substantially rectangular shape. Such a rectangular shape can help maximize the use of available space in a packaging case, in some instances optimizing the maximum quantity of containers that can be transported in a pallet while minimizing transportation costs. The container may be formed via blow molding, or a multilayer, lined, paperboard construction similar to that developed by Tetra Pak International SA, but may have additional features and attributes specifically tailored for use in medical treatments such as an IV treatment or similar. The container may include a graduated, thin-walled, hollow plastic and/or paperboard container with a flush (or recessed) access connector, a handle, and a hanging feature (e.g., for hanging the container on IV poles or the like), where such a container can be used to administer fluids into the human body via IV, or the peritoneal cavity through a catheter, or similar. In some aspects, the robustness and strength of such a hybrid or wholly plastic container can eliminate the need of handling such containers with extra care, and can reduce the quantity of scrap due to damaged product.

The size and dimensions of a container according to the present teachings can vary depending on the prescribed volume of formulated fluid for a particular use case, e.g., a medical therapy and/or treatment. The type of access connector in a container according to the present teachings can also vary; however, in general, an aspect of the present teachings includes a connector that is flush or recessed relative to the bottom of the container to allow the container to rest upright on a surface without external support, and where the connector is protected from damage when so situated (e.g., such that the risk of damage such as a tube kink or a broken connector is mitigated). Such a connector may have a vented or non-vented cap, e.g., depending on the type of sterilization.

A container according to the present teachings may include a handle. The handle may allow for relatively easy handling, use, and lifting of the container when compared to existing bags used for similar purposes such as a medical treatment requiring intravenous and/or dialysis therapy.

Example advantages of aspects of the present teachings may include one or more of the following: the container includes protection for the access connector, preventing damage thereof; the container may be relatively easy to lift and/or hang on an IV pole or similar; the container may be relatively easy to pack/store and may be efficient for packaging, in some instances maximizing packaging space in a carton/case; the container may be relatively easy to handle; the container may eliminate the need of an overwrap or similar; the container may be user-friendly and intuitive to use; the container may be recyclable; and the like.

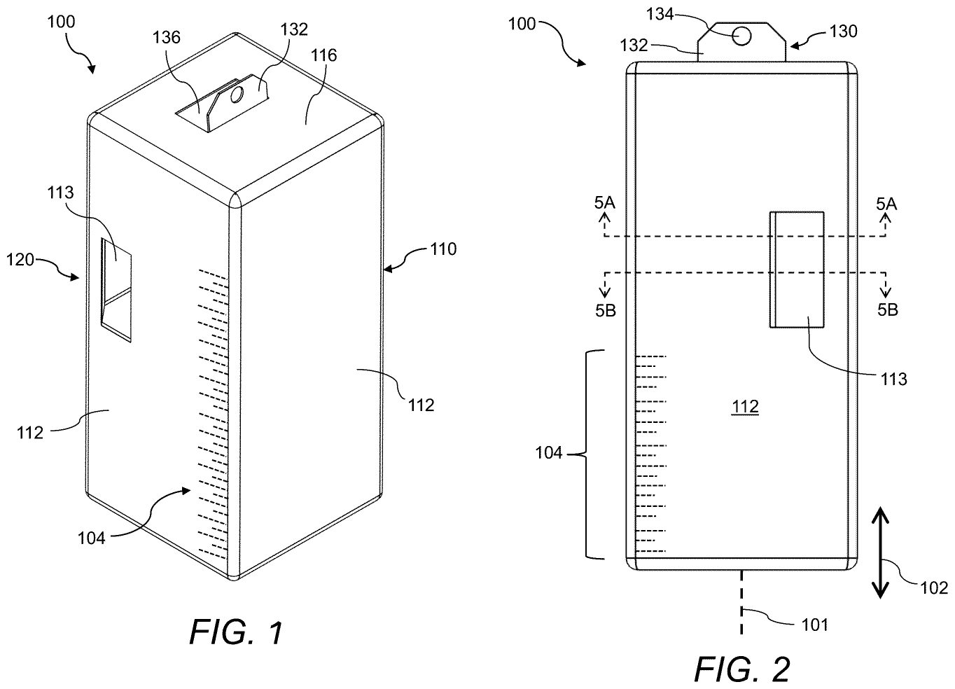

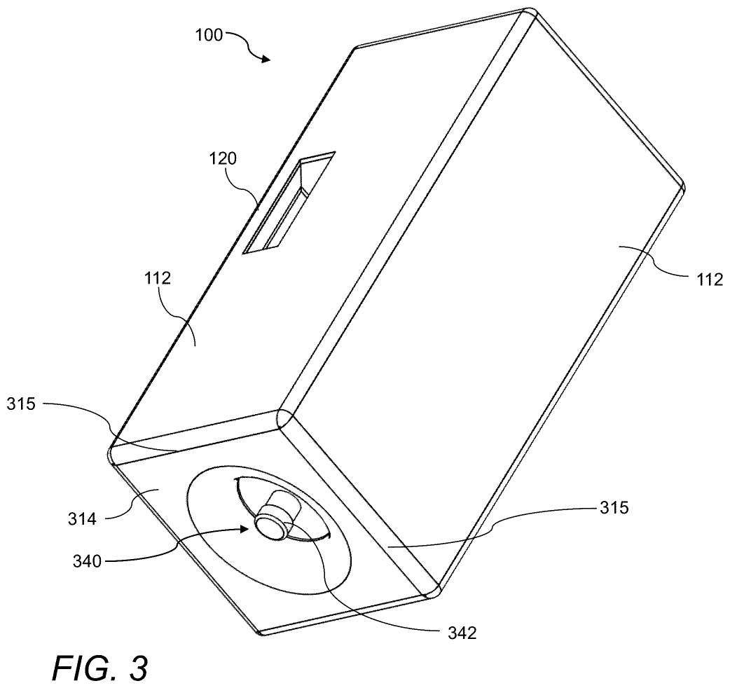

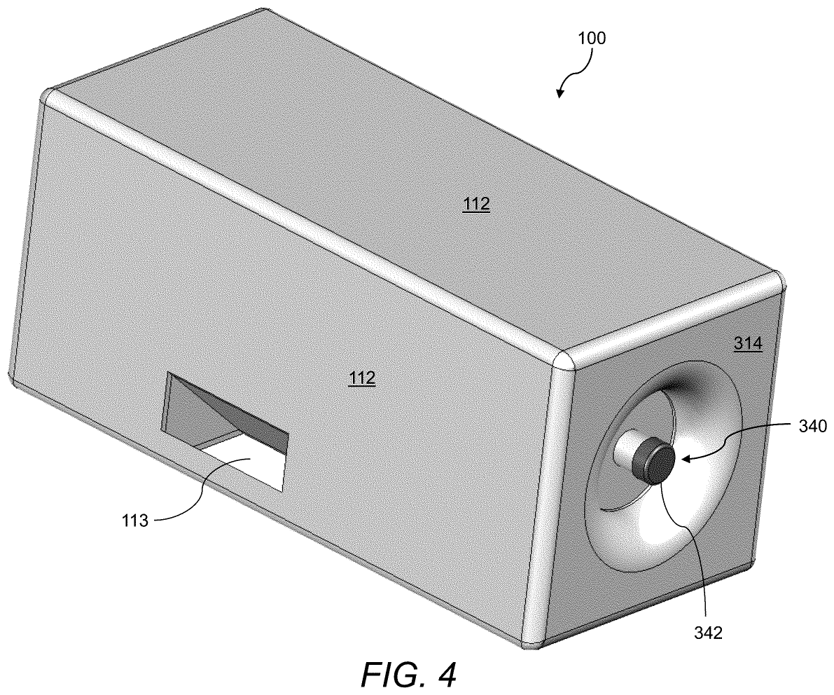

FIG. 1 shows a top, rear perspective view of a container 100 for holding a liquid solution intended for transport into a body of a patient, FIG. 2 shows a front view of the container 100, FIG. 3 shows a bottom perspective view of the container 100, and FIG. 4 shows a side, bottom perspective view of the container 100, all in accordance with representative embodiments. It will be understood that, although the container 100 shown in FIGS. 1-4 contains certain elements as described herein—e.g., a housing 110 having a certain shape, a handle 120, a fixture 130, and a connector 340—other aspects of the present teachings may omit one or more of these elements without departing from the scope of this disclosure.

In general, the container 100 may be structurally configured for use in a dialysis treatment. For example, the container 100 may be configured for use in an automated peritoneal dialysis treatment, continuous ambulatory peritoneal dialysis (CAPD) treatment, and/or a hemodialysis treatment. Also or instead, the container 100 may be structurally configured for use in an intravenous (IV) delivery system.

The general structure of the container 100 may be referred to herein as a housing 110. The housing 110 may be structurally configured to hold a liquid solution without additional internal containers therein—e.g., without inclusion of additional bags or containers disposed within the housing 110. That is, the housing 110 may be hollow but for the liquid contents contained therein. The liquid contents of the housing 110, which may also be referred to as a liquid solution, may be any as described herein. For example, in some aspects, the liquid solution includes a dialysate. In such aspects, the dialysate may include at least one of sodium, calcium, magnesium, chloride, lactate, and the like. Also or instead, the dialysate may include sodium chloride. By way of further example, the liquid solution includes one or more of: a crystalloid solution, a colloid solution, a medication, a nutritional formulation, an electrolyte solution, and the like. In this manner, the liquid solution may be intended for use in an IV treatment, i.e., to administer the liquid solution to a patient via an IV line.

The housing 110 may be hermetically sealed, which shall be understood to mean that the housing 110 is substantially leakproof to maintain the liquid solution within the container 100 when the container 100 is not in use. In some instances, the housing 110 may include a seal that also or instead prevents contaminants from entering the container 100. For example, this may include being completely airtight, preventing the entry of air, gases, or other external contaminants. In this manner, hermetic sealing of the housing 110 can help ensure that the contents of the container 100 are protected from external elements, thus preserving the contents therein.

The housing 110 may be formed by a plurality of substantially planar sidewalls 112, a base 314 (see FIG. 3 for an example of the base 314 and the connector 340), and a top 116. Advantageously, the housing 110 may define a cross-section normal to a central longitudinal axis 101 that approximates a polygon to promote grouping of multiple containers 100 together into a predetermined group shape. By way of example, in the embodiment shown in FIGS. 1-4, the housing 110 has four substantially planar sidewalls 112, and in this manner, the cross-section may approximate rectangle or a square. Further, the example embodiment shown in FIGS. 1-4 has a cross-section that has a substantially consistent shape normal to the longitudinal axis 101 along the length of the housing 110; however, it will be understood that aspects may instead include sidewalls that taper or otherwise include features that can affect the cross-sectional shape of the container 100. Similarly, certain aspects may include more or fewer sidewalls 112, e.g., such that a cross-section through the container 100 may resemble a triangle, pentagon, hexagon, and so forth. Further, certain aspects may include curved sidewalls.

Turning back to the container 100 shown in FIGS. 1-4, the container 100 may include a cross-section that approximates a polygon to enable the grouping of multiple containers 100 together into a predetermined group shape. In certain implementations, the predetermined group shape approximates a rectangular box. This may be particularly advantageous for packing multiple containers 100 together within a carton, box, crate, or the like—e.g., for storage, shipping, and/or other transport. In other words, shipping or storage boxes are often box-shaped (e.g., a cube, cuboid, rectangular prism, and the like), and a container 100 according to the present teachings may be shaped such that multiple containers 100 can be arranged within such a structure to minimize or eliminate wasted space, and/or so that they are aligned in a manner that mitigates damage of the containers 100. However, other shapes are also or instead possible for the predetermined group shape.

The container 100 may include a handle 120. The handle 120 may be formed along one or more of the plurality of substantially planar sidewalls 112. In some aspects, the handle 120 is formed by a cutout 113 in one or more of the plurality of substantially planar sidewalls 112 as shown in the figure. For example, the handle 120 may be formed by cutouts 113 in two adjacent sidewalls 112. As shown in the figures, in some aspects, the cutout 113 may be substantially rectangular. In some aspects, two adjacent cutouts 113 may be, or may not be, connected by a passageway therebetween. In certain embodiments, the handle 120 may be configured for containing liquid contents by being in fluid communication with the remainder of the interior of the housing 110, or the handle 120 may not be in fluid communication with the remainder of the interior of the housing 110. In other aspects, the handle 120 may be a distinct element formed on, or otherwise attached to, the housing 110 (e.g., a sidewall 112, the top 116, and/or the base 314). In such aspects, it may be beneficial to provide a handle 120 that is movable to extend from, and retract back to (e.g., flush with or recessed) such surfaces. That is, the handle 120 may be movable from a storage position where it is flush or recessed relative to an external surface of the housing 110 and an in-use position where it extends from the housing 110. In this manner, in such aspects or otherwise, the maximum dimensions of the cross-section of the housing 110 may not be impacted by the handle 120.

The top 116 of the container 100 may be substantially flat in some aspects, which can be beneficial for the stacking of multiple containers. In other aspects, the top 116 of the container 100 is not flat—e.g., the top 116 of the container 100 may include a triangular or frustoconical apex, similar to a milk carton or the like.

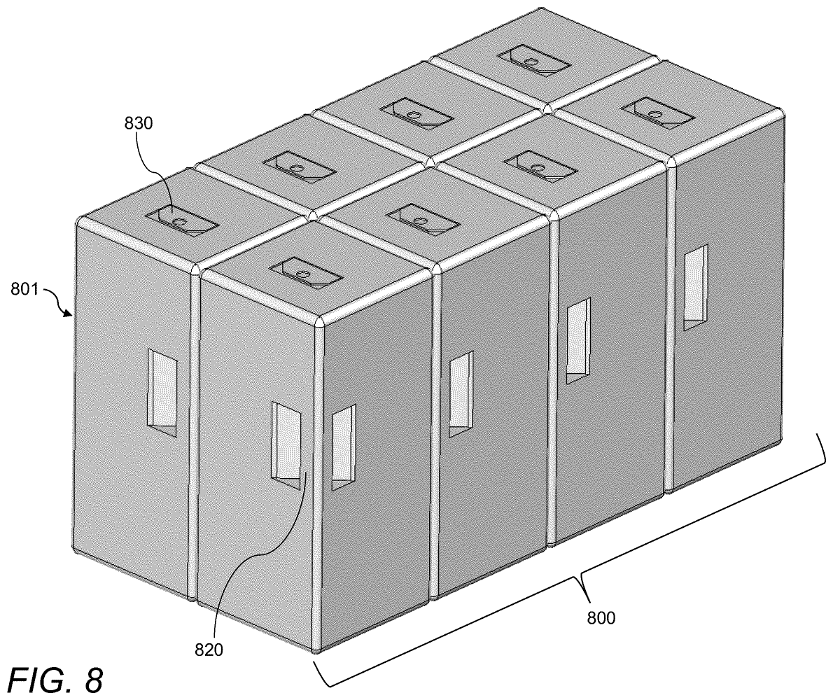

The container 100 may include a fixture 130 disposed on the housing 110, where the fixture 130 is structurally configured for engagement with an external structure (e.g., an IV pole or similar) for hanging the container 100 when in use. As shown in the figures, the fixture 130 may be disposed on the top 116 of the housing 110. For example, and again as shown in the figures, the fixture 130 may include a tab 132 with hole 134. Other means for hanging the container 100 are also or instead possible, including for example one or more of the following: a hook; a chain, rope, cable, or wire; a bracket; a looped end; an adhesive strip or similar; hook and loop; and the like. In some instances, the fixture 130 may be movable from a storage position where it is flush or recessed relative to an external surface of the housing 110 (e.g., the top 116 of the housing 110; see also the fixture 830 of FIG. 8) and an in-use position where it extends from the housing 110. In this manner, the housing 110 may include a recess 136 to accommodate the fixture 130 in the storage position.

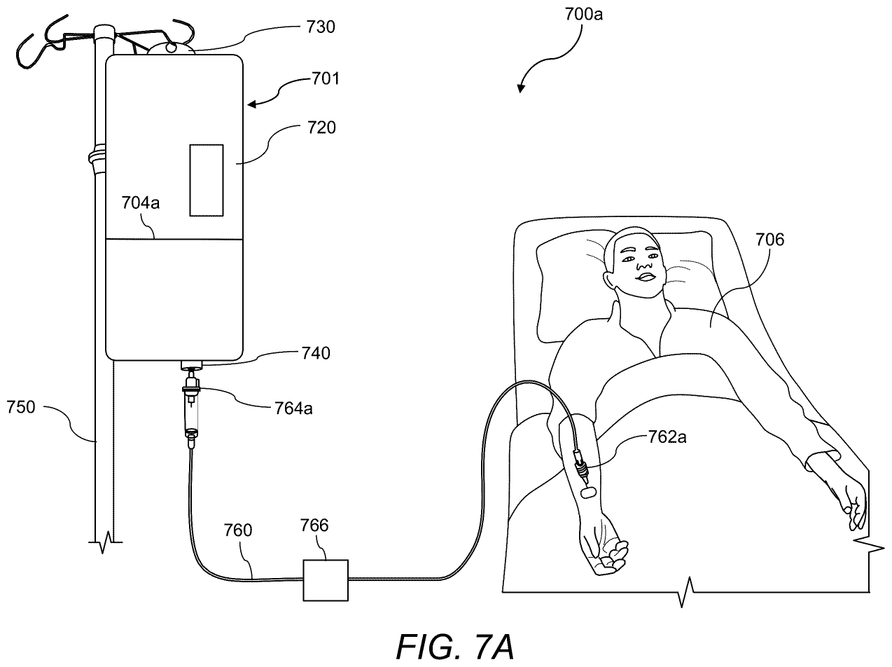

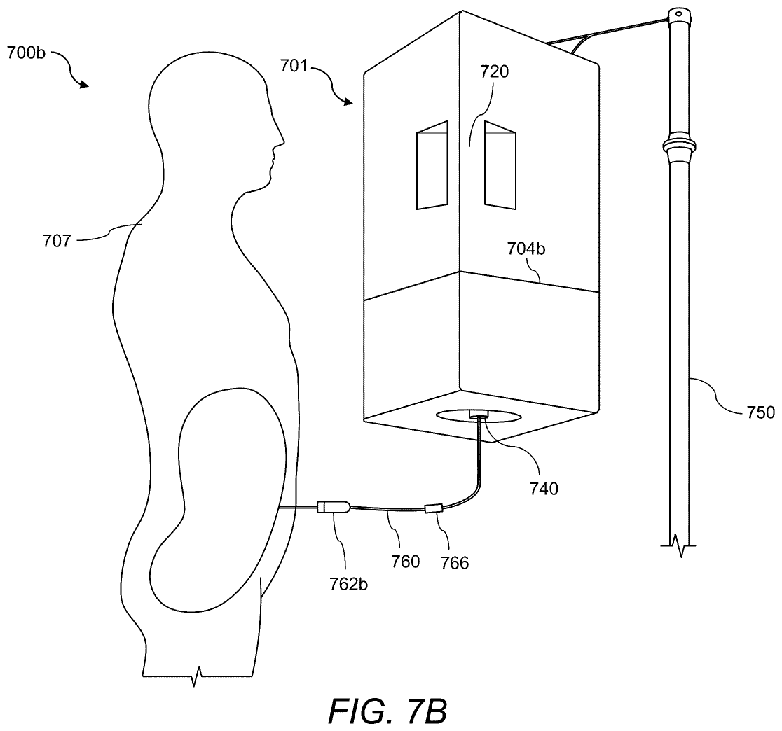

The connector 340 may be disposed on the base 314 of the housing 110. The connector 340 may be structurally configured for engagement with tubing (see, e.g., FIGS. 7A and 7B described below) for draining liquid solution from the container 100 into the tubing. In some aspects, the tubing leads to a catheter for delivery of the liquid solution into a patient's body. Also or instead, the tubing may lead to one or more of an arteriovenous fistula, an arteriovenous graft, a catheter, and the like. In this manner, the connector 340 may be the same as or similar to an IV port or an IV access port, which are configured for attaching to IV tubing (or other tubing) that administers fluids and medications to patients. Thus, the connector 340 may include a port such as a spike port. In this manner, the connector 340 may also or instead include a seal at an end of the port that maintains the sterility of the liquid solution contained therein, where the seal is designed to be punctured by the spike of the IV tubing or similar. The connector 340 may feature a cap 342, which may be a removable sterile cap that protects the port from contamination before use. More generally, the connector 340 may be the connection point for IV tubing or the like, where a tubing spike or the like is inserted into the port to create a secure pathway for fluid to flow from the container 100 into a patient's IV line or other tubing line. In operation, the connector 340 may have its cap 342 removed, the port may be disinfected, and tubing or the like may be coupled thereto, e.g., via a spike or the like. Once connected, the flow of fluids can be regulated through a tubing's flow control mechanisms, such as a roller clamp, an electronic infusion pump, one or more valves and/or restrictors, and the like.

The connector 340 may include a vented cap in some aspects. In other aspects, the connector 340 may include a non-vented cap. In such aspects, the housing 110 may be structurally configured to collapse when the liquid solution is drained from the container 100.

Advantageously, the connector 340, when in a storage position, may project downwardly along a z-axis 102 no further than terminal ends 315 of the plurality of substantially planar sidewalls 112 disposed adjacent to the base 314. Thus, the connector 340 may be flush with the base 314 in an aspect, flush with terminal ends 315 of the plurality of substantially planar sidewalls 112 in an aspect, or recessed with respect to one or more of the base 314 and terminal ends 315 of the plurality of substantially planar sidewalls 112 in an aspect. Similarly, the base 314 itself may be flush with terminal ends 315 of the plurality of substantially planar sidewalls 112 in an aspect (as shown in the figures), or at least partially recessed with respect to terminal ends 315 of the plurality of substantially planar sidewalls 112 in an aspect. For example, in a substantially flush embodiment, the connector 340 may project downwardly along the z-axis 102 no further than junctions between the plurality of substantially planar sidewalls 112 and the base 314. In a recessed embodiment, the base 314 may be at least partially recessed relative to the terminal ends 315 of the substantially planar sidewalls 112—i.e., the junction of the sidewalls 112 and the base 314 may be recessed such that the sidewalls 112 extend further than the base 314 such that their terminal ends form “feet” of the container 100 (not shown).

The connector 340 may be movable between the storage position and a draining position, where the connector 340 projects further downward along the z-axis 102 when in the draining position than in the storage position. For example, a technician or other user may manually pull the connector 340 downward along the z-axis 102 when the container 100 is placed for use and/or when tubing is coupled thereto. Also or instead, the connector 340 may automatically move from the storage position to the draining position when a predetermined engagement with the tubing is present, or otherwise when the container 100 is readied for use. In this manner, the predetermined engagement may include the opening of a valve or the like, and/or the beginning of liquid removal from the container 100 into tubing connected thereto. To this end, the automatic movement of the connector 340 may be caused by a pressure differential from opening a valve or the like, which triggers the connector 340 to drop down in an embodiment.

The base 314 may include a planar surface perpendicular to at least one of the substantially planar sidewalls 112. Also or instead, the base 314 may include one or more sloped surfaces pitched toward the connector 340. Such a sloped surface may be present on the exterior of the container 100 and/or within the interior of the container 100. When disposed within the interior of the container 100, the sloped surface may assist in guiding the liquid contents from within the interior of the container 100 toward the entrance of the connector 340, which can help drain the container 100 fully. The base 314 may include features such that the container 100 remains upright in absence of external forces when placed on a level surface with the connector 340 projecting therefrom along the z-axis 102 (at least while in its storage position), with or without the liquid solution present therein. This may include a base 314 that is substantially flat and/or a base 314 that includes recessed surfaces. In some aspects, both in its storage position and its draining position, the container 100 may remain upright in absence of external forces when placed on a level surface. In such aspects, the connector 340 may be recessed from at least the terminal ends 315 of the sidewalls 112 when in its draining position.

Various manufacturing techniques are possible for making the container 100, including for example, blow molding and/or use of packaging board. For example, in an aspect, the housing 110 is blow molded, e.g., the housing 110 may be at least in part formed of a blow-molded plastic. By way of further example, the housing 110 may also or instead be made from plastic-coated paper. To this end, a coating of the plastic-coated paper may include at least one of: polyethylene, polyolefin, silicone, wax, and the like. Also or instead, the housing 110 may be made from liquid packaging board—e.g., a multilayer packaging board formed of one or more of plastic, paper, and/or metal. Such a liquid packaging board may include layers of polyethylene and paper. Such a liquid packaging board may also or instead include a layer of metal (e.g., aluminum or similar). In such aspects, a paperboard material may provide stability and strength for the housing 110, a polyethylene layer or similar may be used for a moisture/liquid barrier, and a metal layer (e.g., of aluminum foil or similar) may act as a barrier to light, oxygen, and/or bacteria or other contaminants.

In certain example implementations, the housing 110 is at least semitransparent. For example, the housing 110 may be translucent. Having at least some semitransparency in the housing 110 may enable a user to view the contents, and more particularly the level of the contents, within the container 100. To this end, in some aspects, the container 100 may include one or more markings 104 on one or more sidewalls 112, where the markings 104 are related to a volume of the liquid solution within the container 100. That is, the markings 104 may indicate a volume by viewing a marking 104 relative to a level of liquid contents viewable through a sidewall 112 of the housing 110. In other aspects, the housing 110 may be, at least in part, substantially opaque.

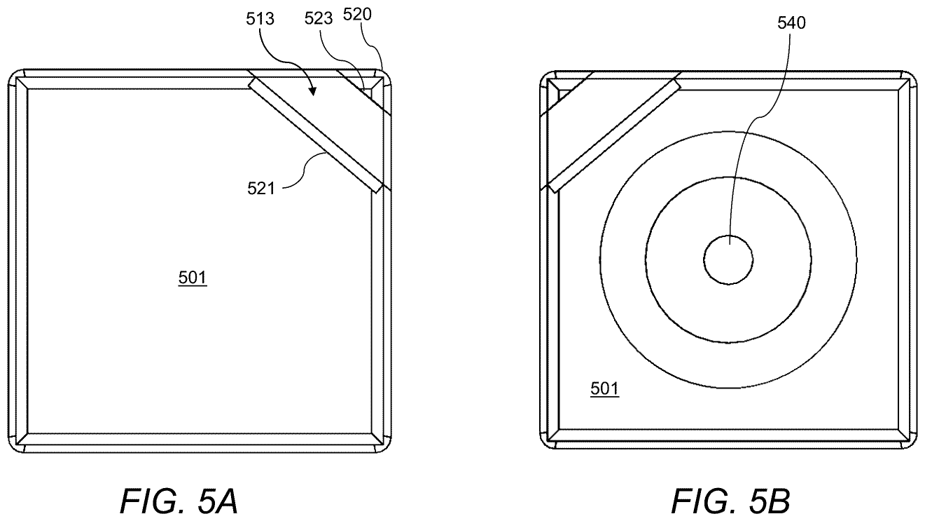

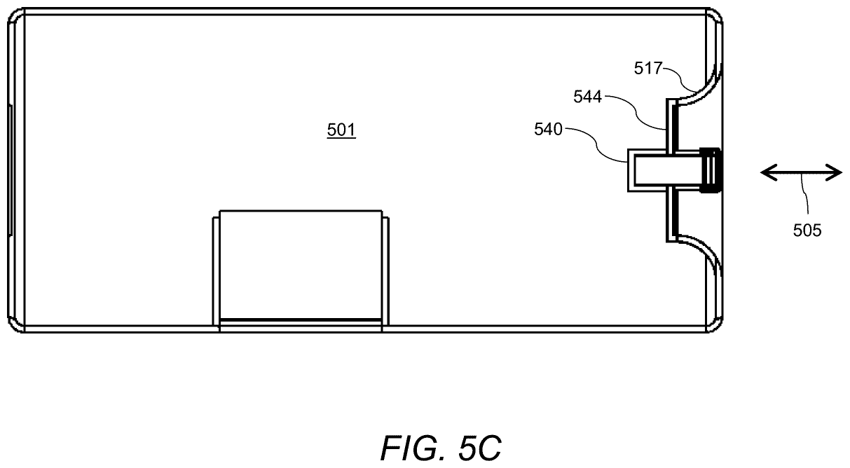

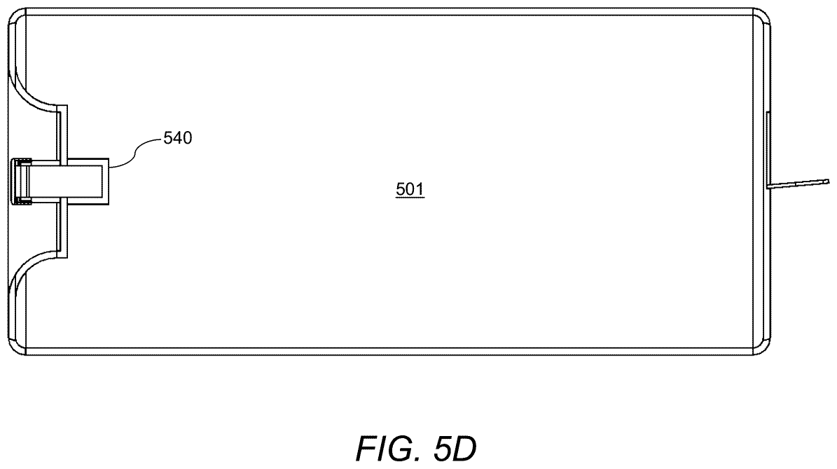

FIGS. 5A-5D show cross-sectional views through a container, in accordance with a representative embodiment. Specifically, FIGS. 5A and 5B shows cross-sectional views through section 5A-5A and section 5B-5B of the container 100 of FIG. 2, respectively, i.e., normal to the central longitudinal axis 101; and FIGS. 5C and 5D show cross-sectional views lengthwise (i.e., along the central longitudinal axis 101) in opposite directions through the container 100. These drawings are provided to demonstrate and emphasize that the housing 110 of the container 100 of FIGS. 1-4 above may define a cross-section that approximates a polygon to promote grouping of multiple devices together into a predetermined group shape. For example, the cross-section of FIGS. 5A and 5B approximates a rectangle (or more particularly for this embodiment, a square), and the cross-section of FIGS. 5C and 5D approximates a rectangle.

It shall be understood that the embodiment shown in these cross-sections, by way of example, includes a truncated portion 521 within the interior 501 of the container to accommodate a handle 520, which may be formed by a cutout 513 through two sidewalls of the container. Further, this example embodiment includes a hollow handle portion 523 that liquid contents can traverse therethrough. It shall be noted, though, that the handle 520 does not impact the outer shape/perimeter of the cross-section that defines a polygon (i.e., rectangle) in this embodiment. However, it will be understood that there are many different configurations of a handle that can be included on containers as described herein without departing from the scope of this disclosure—e.g., where the handle does not affect the shape or volume of the interior 501 of the container, such that the interior 501 maintains a substantially consistent shape throughout the container—and that handles can be completely omitted in some embodiments of the containers of the present teachings. It should further be noted that the interior 501 of this example embodiment is devoid of any internal containers, bags, etc., which may be advantageous for production of the container—e.g., by using less material.

FIG. 5B shows a cross-section looking toward the connector 540 from the interior 501 of the container. The circular shapes surrounding the connector 540 may represent a portion of the connector 540, expandable joints such that the connector 540 is movable (e.g., between a storage position to a draining position, where the draining position is one in which the connector 540 is extended away from the housing of the container), and/or pitched surfaces for directing the flow of liquid contents of the container. In some instances, the connector 540 is flush within the interior 501 of the container; in other instances, the connector 540 projects into the interior 501 of the container at least when disposed in its storage position. For example, FIGS. 5C and 5D show embodiments where the connector 540 projects into the interior 501 of the container, at least when disposed in its storage position. These figures further show an example aspect where a portion of the connector includes a flat surface 544 that may move with the connector 540 (and in some cases is integral with the connector 540) via an expandable portion 517 of the housing (e.g., a portion of the base of the housing) that allows for movement of the connector 540 relative to the housing between a storage position (shown in FIGS. 5C and 5D) and a drainage position where it extends from the housing—i.e., in one or more of the directions of the arrows 505 shown in FIG. 5C.

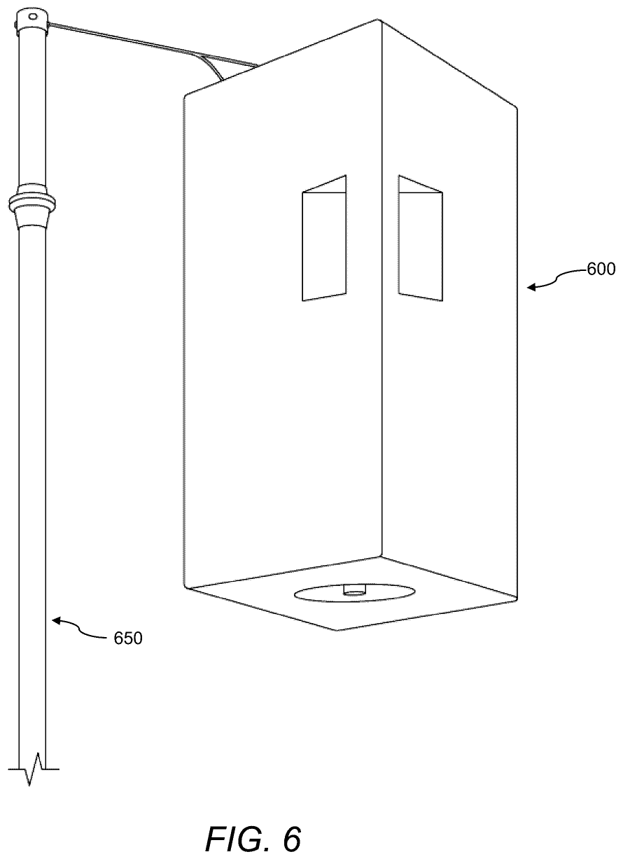

FIG. 6 shows a container in use or readied for use, in accordance with a representative embodiment. The container 600 may be the same as, or similar to, the container described with reference to FIGS. 1-4 above. The container 600 in this figure is shown ready for use—i.e., hung from or otherwise mounted to an IV pole 650, where it can be connected to tubing for transferring its liquid contents from the container 600 into a patient's body.

FIGS. 7A and 7B show fluid delivery systems, in accordance with representative embodiments. Specifically, the system 700a of FIG. 7A shows a fluid delivery system in the form of an IV treatment by way of example, and the system 700b of FIG. 7B shows a fluid delivery system in the form of a dialysis treatment (e.g., a CAPD treatment) by way of example. It will be understood that one or more components of the system 700a of FIG. 7A may also or instead be included in the system 700b of FIG. 7B, and vice-versa. For example, each of these systems 700a, 700b may include a container 701 that holds a liquid solution 704a, 704b therein, a support structure 750, tubing 760, and various connectors and control elements such as those commonly used in an IV system, a dialysis system, and the like.

The liquid solution 704a, 704b may be the same as or similar to any of the possible contents of the containers described herein. In general, the liquid solution 704a, 704b may be configured for transmission into a body of a patient 706, 707. For example, the liquid solution 704a in the system 700a of FIG. 7A may be configured for transmission into the body through a vein or similar, and the liquid solution 704b in the system 700b of FIG. 7B may be configured for transmission into the body through a catheter or the like surgically installed in the peritoneal cavity or similar. In this manner, each of the systems 700a, 700b may include a patient connector 762a, 762b for coupling the tubing 760 to the patient 706. One or more of the patient connectors 762a, 762b may include any as known in the art, such as one or more of a catheter, an arteriovenous fistula, an arteriovenous graft, and the like. One or more of the patient connectors 762a, 762b may also or instead include one or more of a needle, a Luer lock connector or similar, a rubber bulb, a clamp, a cannula, extension tubing, and the like.

By way of example, the liquid solution 704a may include a saline solution, medication, or the like; and the liquid solution 704b may include a dialysate, such as a solution including one or more of sodium, calcium, magnesium, chloride, lactate, and similar. One or more of the liquid solutions 704a, 704b may also or instead include one or more of a crystalloid solution, a colloid solution, a medication, a nutritional formulation, an electrolyte solution, and similar.

The container 701 may be the same as or similar to any of the containers described herein. In some aspects, the container 701 in each of the systems 700a, 700b is the same, e.g., except for their respective liquid solution 704a, 704b contents. In other aspects, the containers in the systems 700a, 700b may be different—e.g., having a different size to hold a different volume of contents, and/or having other differing characteristics such as any of the attributes of containers as described herein. In certain implementations of either system 700a, 700b, the container 701 may include a housing formed by a plurality of substantially planar sidewalls, a base, and a top, where the housing holds the liquid solution 704a, 704b without additional internal containers therein, the housing is hermetically sealed, and the housing defines a cross-section normal to a central longitudinal axis that approximates a polygon to promote grouping of multiple containers together into a predetermined group shape. The container 701 may further include a handle 720, e.g., formed along one or more of the plurality of substantially planar sidewalls. The container 701 may further include a fixture 730 disposed on the housing that is structurally configured for engagement with an external structure for hanging the container when in use—e.g., the support structure 750. The container 701 may further include a connector 740 disposed on the base of the housing and structurally configured for engagement with tubing 760 for draining the liquid solution 704a, 704b from the container 701 into the tubing 760. In some aspects, the connector 740, when in a storage position, projects downwardly along a z-axis no further than terminal ends of the plurality of substantially planar sidewalls disposed adjacent to the base. The connector 740 in FIG. 7A is shown by way of representation projecting further than the terminal ends of the plurality of substantially planar sidewalls. That is, the connector 740 is shown in a draining position, where the connector 740 projects further downward along the z-axis when in this draining position.

The support structure 750 may be any external structure for hanging the container 701 when in use. For example, the support structure 750 may include an IV pole or the like. The support structure 750 may also or instead include a piece of medical equipment.

The tubing 760 may be any as known in the art, e.g., for use in an IV treatment, a dialysis treatment, and similar. The tubing 760 itself may feature one or more of the various connectors and control elements, or these may be independent elements for engagement with the tubing 760 or other parts of the system 700a, 700b. The various connectors and control elements may include one or more of the following: the patient connector 762a, 762b, a solution connector 764a, flow controls 766, clamps, and the like, such as any similar components used in an IV system, a dialysis system, and the like.

The solution connector 764a may be structurally configured for engagement with the connector 740 of the container 701. In some instances, the solution connector 764a is the same as or similar to any commonly used in an IV system, a dialysis system, or similar, such that the container 701 can be connected to existing medical equipment. In this manner, the solution connector 764a may include one or more of the following: a protective cap (for removal before connecting the solution connector 764a to the connector 740 of the container 701), a spike connector, an air filter, an air vent, a drip chamber, a solution filter, one or more additional ports, a check valve and/or other valves, a clamp, and the like.

The flow controls 766 may be structurally configured for engagement with the tubing 760, directly or indirectly. The flow controls 766 may include any commonly used in an IV system, a dialysis system, or similar. In this manner, the flow controls 766 may include one or more of the following: a clamp (e.g., a slider clamp, a roller clamp, and similar), another port, a valve, a restrictor, and the like.

To use one or more of the systems 700a, 700b, a user (e.g., a medical technician, the patient 706, 707, or another) may retrieve a container 701, e.g., from a package within which multiple containers can be efficiently stored without wasting space (in contrast to the wasted space common to present methods of storing IV bags or dialysate bags) and similar. In some aspects, the fixture 730 of the container 701 may be disposed in a storage position where it is flush or recessed relative to an external surface of the container 701. In such aspects, the user may move the fixture 730 such that it is disposed in an in-use position where it extends from the container 701 and/or where it otherwise is released from its storage position. A user may lift or otherwise move the container 701 via its handle 720—which may provide for easier mobility of the container 701 relative to IV bags, dialysate bags, and the like—and engage the container 701 with an external structure (e.g., the support structure 750 such as an IV pole or similar) for use. Engaging the container 701 with a support structure 750 may include hanging the container 701, via the fixture 730, from the support structure 750. This can involve placing a hook or other projection of the support structure 750 through a hole in the fixture 730. In alternative aspects, the handle 720 can be used as the fixture for hanging the container 701. Once readied for use by engagement with the support structure 750, the user may prepare the connector 740 of the device for engagement with tubing 760. In some aspects, this includes removal of a cap and engaging the tubing 760 with the connector 740 of the container 701 via a solution connector 764a. This may also or instead include moving the connector 740 of the container 701 from a storage position where it is flush or recessed relative to an external surface of the container 701 (e.g., along the base) to an in-use position where it extends from the container 701 or is otherwise released from its storage position. In some instances, movement from the storage position to the in-use position occurs automatically, e.g., when engagement with the tubing 760 is initiated or completed, and/or when the liquid solution 704a, 704b is released into the tubing 760. The user may engage the tubing 760 with the patient 706, 707, e.g., using the patient connector 762a, 762b. Transmission of the liquid solution 704a, 704b from the container 701 through the tubing 760 to the patient 706, 707 may then be initiated, where a flow of the liquid solution 704a, 704b may be controlled, e.g., using one or more flow controls 766 or the like. When use is complete, the container 701 may be discarded, recycled, reused, and so on. This process may also involve sterilization of the container 701 or a component thereof (or another component of the system 700a, 700b), before, during, or after use.

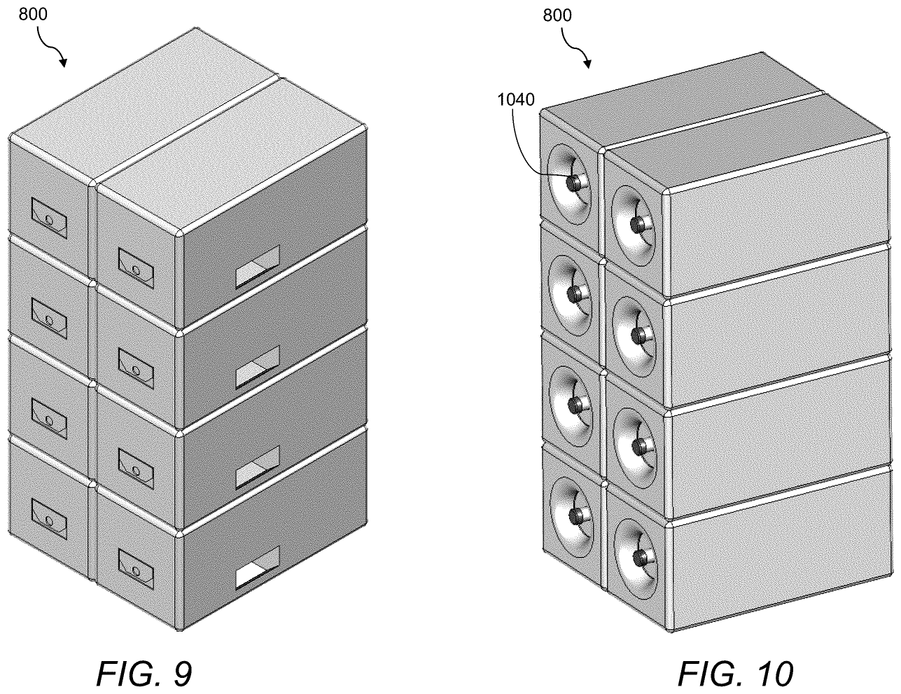

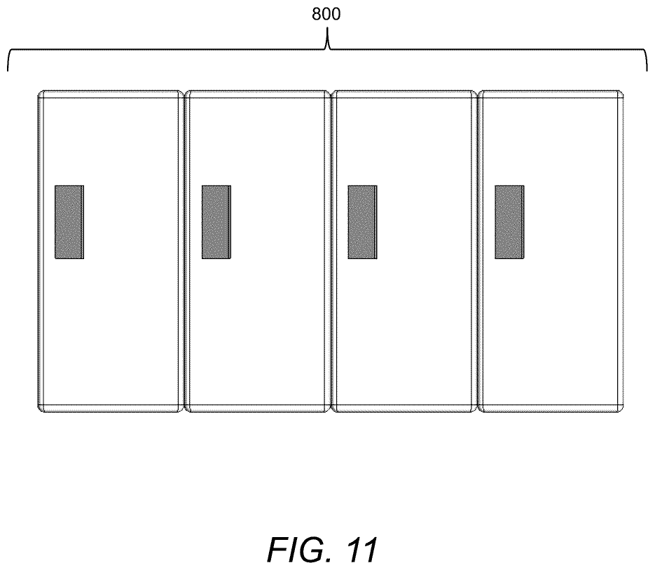

FIGS. 8-11 show a plurality of containers, in accordance with representative embodiments. That is, these figures show an example of eight containers 801 that could be packaged in a case (e.g., a box for storage and/or transport) for maximizing the use of space in the case. In this manner, these figures show example containers 801 according to the present teachings that are arranged in a predetermined group shape 800 approximating, by way of example, a rectangular box. Other predetermined group shapes 800 are also or instead possible.

As shown in the figures, one or more of the handle 820, the fixture 830, and the connector 1040 of the containers 801 may be structurally configured such that they do not project from the housing of the container 801. In this manner, the housing of the containers 801 may define a maximum cross-section that approximates a polygon to promote grouping of multiple containers together into the predetermined group shape 800, regardless of the presence of the handle 820, the fixture 830, and the connector 1040. One or more of the handle 820, the fixture 830, and the connector 1040 may be situated in a storage position in these figures, where they can be moved to an in-use position for using the containers 801 as described herein (e.g., with reference to FIGS. 7A and 7B).

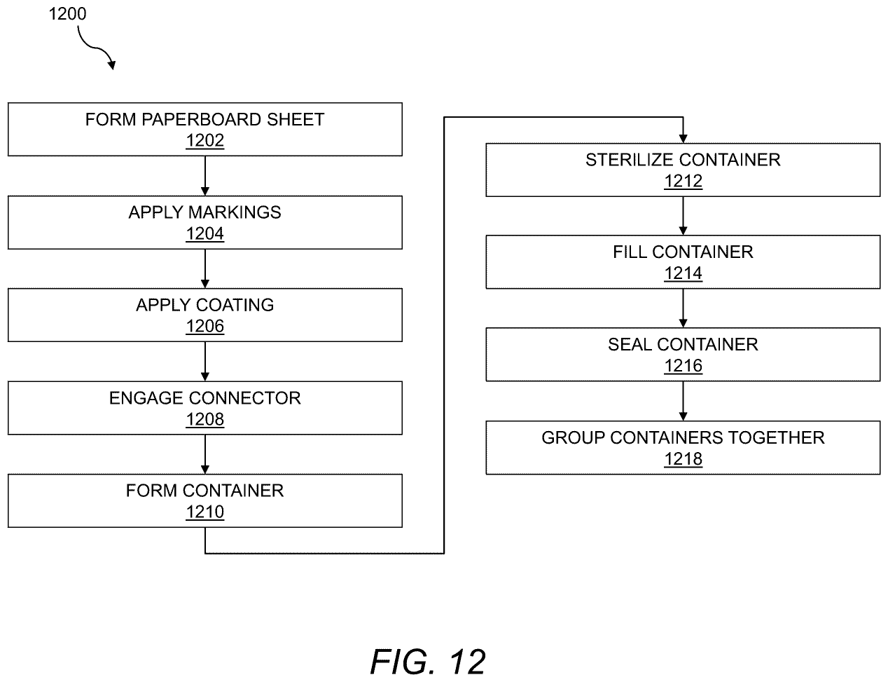

FIG. 12 is a flow chart of a method of manufacturing a container, in accordance with a representative embodiment. The method 1200 may be performed to create one or more of the devices and systems described herein. It will be understood that this method 1200 is shown by way of example, and the containers described herein may be manufactured in a different manner, e.g., via blow molding or similar.

As shown in step 1202, the method 1200 may include forming a paperboard sheet. In some aspects, this may include cutting a paperboard sheet into a predetermined shape. This may also or instead include bending a sheet in preparation for folding.

As shown in step 1204, the method 1200 may include applying markings to the paperboard sheet. For example, this may include printing one or more markings onto the sheet.

As shown in step 1206, the method 1200 may include applying a coating to the paperboard sheet, e.g., to create a waterproof and/or leakproof sheet. The coating may include a plastic. The coating may also or instead include at least one of: polyethylene, polyolefin, silicone, wax, and the like. The coating may be applied via one or more layers of material added to the paperboard. By way of example, one or more of these layers of material added to the paperboard may include polyethylene, a metal (e.g., aluminum, such as aluminum foil), and the like.

As shown in step 1208, the method 1200 may include engaging a connector, e.g., a connector structurally configured for engagement with tubing. By way of example, a connector may be engaged with a portion of the base. The connector may be engaged in such a manner so that the connector, when in a storage position, projects downwardly along a z-axis no further than terminal ends of a plurality of substantially planar sidewalls disposed adjacent to the base.

As shown in step 1210, the method 1200 may include forming the container. For example, this may include forming the waterproof sheet into the container. In some instances, this may include folding the sheet into the shape of the container—e.g., forming the waterproof sheet into the container may include folding the waterproof sheet into the container having a maximum cross-section that approximates the polygon. In some instances, this step may include perforating, scoring, compressing, or similar the waterproof sheet along edges for folding the sheet. In some instances, this may include cutting the sheet into discreet elements for joining to form the shape of the container. The container may be formed to include substantially planar sidewalls, a top, and a base. The container may define a maximum cross-section normal to a central longitudinal axis that approximates a polygon to promote grouping of multiple containers together into a predetermined group shape as described herein. The container may be formed to further include one or more of a handle formed along one or more of the plurality of substantially planar sidewalls, a fixture structurally configured for engagement with an external structure for hanging the container when in use, and a portion of the base formed for engagement with the connector.

As shown in step 1212, the method 1200 may include sterilizing the container. This may include one or more of the following: heat treatment, light treatment, hydrogen peroxide treatment, and the like.

As shown in step 1214, the method 1200 may include filling the container with a liquid solution. In some aspects, this may include an aseptic filling process to maintain a sterile environment to prevent contamination. The liquid solution may be sterilized separately from the container, where it is filled into the container under aseptic conditions to ensure that both the container and the liquid solution are free from any unwanted contaminants (e.g., unwanted microorganisms and the like). In some aspects, filling the container with the liquid solution happens while forming the container, or before completing formation thereof.

As shown in step 1216, the method 1200 may include sealing the container. This may occur immediately after filling, within the aseptic environment, to maintain sterility. This may be done using heat sealing or ultrasonic sealing methods, ensuring a tight and sterile closure.

As shown in step 1218, the method 1200 may include grouping multiple containers together into the predetermined group shape. As discussed herein, in an aspect, the predetermined group shape approximates a rectangular box. The predetermined group shape may be packaged in a case—e.g., a box intended for transport.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings.

Unless the context clearly requires otherwise, throughout the description, the words “comprise,” “comprising,” “include,” “including,” and the like are to be construed in an inclusive sense as opposed to an exclusive or exhaustive sense; that is to say, in a sense of “including, but not limited to.” Additionally, the words “herein,” “hereunder,” “above,” “below,” and words of similar import refer to this application as a whole and not to any particular portions of this application.

It will be appreciated that the devices, systems, and methods described above are set forth by way of example and not of limitation. Absent an explicit indication to the contrary, the disclosed steps may be modified, supplemented, omitted, and/or re-ordered without departing from the scope of this disclosure. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the implementations described herein are intended to include any suitable method of causing such method steps to be performed, consistent with the patentability of the following claims, unless a different meaning is expressly provided or otherwise clear from the context. So, for example performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A container for holding a liquid solution intended for transport into a body of a patient, the container comprising:
a housing formed by a plurality of substantially planar sidewalls, a base, and a top, wherein:
the housing is structurally configured to hold the liquid solution without additional internal containers therein,
the housing is hermetically sealed, and
the housing defines a cross-section normal to a central longitudinal axis that approximates a polygon to promote grouping of multiple containers together into a predetermined group shape;
a handle formed along one or more of the plurality of substantially planar sidewalls;
a fixture disposed on the housing, the fixture structurally configured for engagement with an external structure for hanging the container when in use; and
a connector disposed on the base of the housing and structurally configured for engagement with tubing for draining the liquid solution from the container into the tubing, wherein the connector, when in a storage position, projects downwardly along a z-axis no further than terminal ends of the plurality of substantially planar sidewalls disposed adjacent to the base.

2. The container of claim 1, wherein the housing has four substantially planar sidewalls.

3. The container of claim 2, wherein the cross-section approximates a rectangle.

4. The container of claim 1, wherein the predetermined group shape approximates a rectangular box.

5. The container of claim 1, wherein the housing is at least semitransparent.

6. The container of claim 5, further comprising one or more markings on one or more sidewalls, the one or more markings related to a volume of the liquid solution within the container.

7. The container of claim 1, wherein the container remains upright in absence of external forces when placed on a level surface with the connector projecting along the z-axis, with or without the liquid solution present therein.

8. The container of claim 1, wherein the housing is hollow but for the liquid solution.

9. The container of claim 1, wherein the housing is made from plastic-coated paper.

10. The container of claim 1, wherein the housing is made from liquid packaging board.

11. The container of claim 1, wherein the housing is blow molded.

12. The container of claim 1, wherein the connector projects downwardly along the z-axis no further than junctions between the plurality of substantially planar sidewalls and the base.

13. The container of claim 1, wherein maximum dimensions of the cross-section of the housing are not impacted by the handle.

14. The container of claim 1, wherein the fixture is movable from a storage position where it is flush or recessed relative to an external surface of the housing and an in-use position where it extends from the housing.

15. The container of claim 1, wherein the connector is movable between the storage position and a draining position, and wherein the connector projects further downward along the z-axis when in the draining position.

16. The container of claim 1, wherein the connector includes a vented cap.

17. The container of claim 1, wherein the tubing is part of an intravenous delivery system.

18. The container of claim 1, wherein the container is configured for use in a dialysis treatment.

19. A system, comprising:
a liquid solution configured for transmission into a body of a patient; and
a container comprising:
a housing formed by a plurality of substantially planar sidewalls, a base, and a top, wherein:
the housing holds the liquid solution without additional internal containers therein,
the housing is hermetically sealed, and
the housing defines a cross-section normal to a central longitudinal axis that approximates a polygon to promote grouping of multiple containers together into a predetermined group shape;
a handle formed along one or more of the plurality of substantially planar sidewalls;
a fixture disposed on the housing, the fixture structurally configured for engagement with an external structure for hanging the container when in use; and
a connector disposed on the base of the housing and structurally configured for engagement with tubing for draining the liquid solution from the container into the tubing, wherein the connector, when in a storage position, projects downwardly along a z-axis no further than terminal ends of the plurality of substantially planar sidewalls disposed adjacent to the base.

20. A method of manufacturing a container, comprising:
forming a paperboard sheet;
applying a coating to the paperboard sheet to create a waterproof sheet;
forming the waterproof sheet into the container comprising:
substantially planar sidewalls, a top, and a base, wherein the container defines a cross-section normal to a central longitudinal axis that approximates a polygon to promote grouping of multiple containers together into a predetermined group shape;
a handle formed along one or more of the substantially planar sidewalls;
a fixture structurally configured for engagement with an external structure for hanging the container when in use; and
a portion of the base formed for engagement with a connector structurally configured for engagement with tubing;
filling the container with a liquid solution; and
sealing the container.
